(19) United States
(12) Patent Application Publication
(10) Pub. No.: US 2026/0325294 A1
(43) Pub. Date: Oct. 22, 2026

(54) BALLOONS FOR MEDICAL DEVICES

(71) Applicant: Edwards Lifesciences Corporation, Irvine, CA (US)

(72) Inventors: Ilan Leshecz, Hadera (IL); Aviv Geva, Hod Hasharon (IL)

(73) Assignee: Edwards Lifesciences Corporation, Irvine, CA (US)

(21) Appl. No.: 19/697,322

(22) Filed: Jun. 3, 2026

Related U.S. Application Data
(63) Continuation of application No. PCT/US2024/058878, filed on Dec. 6, 2024.
(60) Provisional application No. 63/612,824, filed on Dec. 20, 2023.

(51) Int. Cl. A61F 2/24

(52) U.S. Cl. CPC A61F 2/2433; A61F 2/2418

(57) ABSTRACT

Delivery apparatus for a prosthetic device, delivery assemblies, and methods of implanting a prosthetic device. The delivery apparatus includes a shaft, an inflatable balloon mounted on a distal end portion of the shaft, and a restraining assembly. A radially expandable prosthetic device, such as a prosthetic valve, can be mounted over a central portion of the balloon in a radially compressed state. The balloon can be inflated to radially expand the prosthetic device. The restraining assembly can include proximal and distal end caps disposed over the proximal and distal end portions of the balloon and tethers extending the proximal and distal end caps. The tethers can limit movement of the end caps away from each other during inflation of the balloon and thereby limit axial elongation of the balloon. In some examples, the delivery apparatus further include a plurality of axial ribs attached to the center portion of the balloon.

### CROSS-REFERENCE TO RELATED APPLICATIONS

This application is a continuation of PCT Application No. PCT/US2024/058878, filed Dec. 6, 2024, which claims the benefit of U.S. Provisional Patent Application No. 63/612,824, filed Dec. 20, 2023, the entire contents of each of which are incorporated herein by reference.

### FIELD

The present disclosure relates to inflatable balloons for medical devices, for example, apparatus and methods for delivering and implanting implantable, radially expandable prosthetic valves or other implantable devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted so that the prosthetic heart valve can self-expand to its functional size.

### SUMMARY

Described herein are prosthetic heart valves, delivery apparatus, delivery assemblies, and methods for implanting prosthetic heart valves. The disclosed, delivery assemblies and delivery apparatus for prosthetic heart valves, and methods associated methods can, for example, provide improved inflation of a balloon and/or improved radial expansion of a prosthetic heart valve. In some examples, the delivery apparatus disclosed herein can include one or more restraining assemblies that restrict, restrain, and/or limit axial elongation of a balloon. In some examples, such restraining mechanisms can encourage and/or direct inflation fluid to fill a central portion of the balloon (having a radially compressed prosthetic valve mounted therearound) so that delay or lag of inflation of the center portion relative to proximal and distal end portions of the balloon (which are uncovered or exposed from the compressed prosthetic valve) can be reduced. In other words, inflation of the center portion of the balloon can be more even and/or balanced relative to the proximal and distal end portions of the balloon. Further, in some examples, such restraining mechanisms can improve radial expansion of the prosthetic valve by causing radial expansion of a center portion of the prosthetic valve in a more even and predictable manner with the inflow and outflow ends of the prosthetic valve. Further still, in some examples, at a higher end of the working range of the prosthetic valve, such restraining mechanisms can improve detection of fluid pressure indicative of contact between the radially expanded prosthetic valve and a native annulus. As such, the devices and methods disclosed herein can, among other things, overcome one or more of the deficiencies of typical prosthetic heart valves and their delivery apparatus.

A medical apparatus can include a handle and one or more shafts coupled to the handle.

In some examples, the medical apparatus can be a delivery apparatus for a prosthetic implant.

In some examples, the delivery apparatus can include a balloon mounted on a distal end portion of one of the shafts.

In some examples, the prosthetic implant can be a prosthetic heart valve radially compressed around a center portion of the balloon.

In some examples, the delivery apparatus can include a restraining assembly for limiting, restricting, and/or restraining axial elongation of the balloon during inflation thereof.

In some examples, a restraining assembly can include a proximal end cap extending over a proximal portion of the balloon and a distal end cap extending over a distal portion of the balloon.

In some examples, a restraining assembly can include a proximal end cap formed in a proximal portion of the balloon and a distal end cap formed in a distal portion of the balloon.

In some examples, the end caps are formed from a same material as the balloon.

In some examples, the end caps and the balloon are formed from one or more of various polyamides or co-polyamides, such as nylon (for example, nylon 12), Pebax®, Grilamid L25, or silicone and/or other materials or combinations thereof.

In some examples, the end caps have a greater thickness than a wall of the balloon.

In some examples, the thickness of the wall of the balloon is in a range of 0.03 mm to 0.15 mm.

In some examples, the thickness of each of the proximal and distal end caps is in a range of 0.04 mm to 0.2 mm.

In some examples, the end caps are formed from a different material from the balloon, which can have a higher stiffness modulus than the material of the balloon.

In some examples, the end caps are formed from one or more of a polyethylene terephthalate (also known as PET), polybutylene terephthalate, or thermoplastic elastomer copolyester and/or other materials or combinations thereof, or a braided, woven, or knitted structure, such as a fabric comprised of yarns or wires.

In some examples, a restraining assembly can include a plurality of tethers that extend from the proximal end cap to the distal end cap.

In some examples, the plurality of tethers can be attached and/or coupled to or can be integrally formed with and extend from a perimeter portion of each of the end caps.

In some examples, the plurality of tethers can extend over an exterior surface of the balloon.

In some examples, the plurality of tethers can be angularly spaced around the perimeter portions of the end caps.

In some examples, the plurality of tethers can be angularly spaced around a perimeter of the balloon.

In some examples, the plurality of tethers can be attached and/or coupled to or can be integrally formed with and extend from a center portion of each of the end caps.

In some examples, the plurality of tethers can extend along the shaft inside of the balloon.

In some examples, the plurality of tethers can be angularly spaced around a perimeter of the shaft.

In some examples, in a delivery configuration of the delivery apparatus, the tethers can be in a relaxed state.

In some examples, in a deployment configuration of the delivery apparatus, the tethers can be in a taut state.

In some examples, in a deployment configuration of the delivery apparatus, the tethers can be in a taut state.

In some examples, in a deployment configuration of the delivery apparatus, the tethers can prevent or limit movement of the end caps away from each other, and thereby restrain, restrict, and/or limit axial elongation of the balloon beyond a selected or desired distance.

In some examples, a length of the tethers can be selected to define the permitted axial elongation distance and/or the degree of permitted axial elongation of the inflated balloon.

In some examples, a restraining assembly can include a plurality of axial members or ribs angularly spaced around a circumference of the balloon.

In some examples, the plurality of axial members can add structural support, strength, and/or rigidity to the central portion.

In some examples, a restraining assembly can enable inflation of the center portion of the balloon at an earlier phase of the inflation process relative to delivery apparatus lacking the restraining assembly.

In some examples, a restraining assembly can reduce lag or delay of expansion of the central portion relative to the proximal and distal portions of the balloon.

In some examples, a restraining assembly can enable more even (uniform) inflation of the proximal, distal, and center portions of the balloon relative to delivery apparatus lacking a restraining assembly.

An assembly can comprise a prosthetic implant and a delivery apparatus.

In some examples, the prosthetic implant can be a prosthetic heart valve radially compressed around a center portion of a balloon of the delivery apparatus.

In some examples, the delivery apparatus is configured such that inflation of the balloon causes radial expansion of the prosthetic valve.

In some examples, the prosthetic heart valve can include an annular frame and a valve structure disposed within the frame.

In some examples, the prosthetic heart valve can further comprise one or more of the components and/or features discussed herein or other components and/or features.

In some examples, the delivery apparatus can include one or more of the features in the above examples, such as, for example, a restraining assembly for limiting, restricting, and/or restraining axial elongation of the balloon during inflation thereof.

In some examples, a restraining assembly can enable more symmetrical radial expansion of the prosthetic valve when transitioning from the radially compressed state to the radially expanded state relative to a delivery assembly lacking the restraining assembly.

In some examples, a restraining assembly can enable improved utilization of fluid pressure within the balloon to identify contact between the radially expanded prosthetic valve and a native annulus over the working range of the balloon and prosthetic valve (for example, improved utilization of fluid pressure a higher end of the working range) relative to a delivery assembly lacking the restraining assembly.

A method can include introducing a distal end portion of the delivery apparatus into vasculature of a patient and advancing the distal end portion of the delivery apparatus toward a native heart valve of the patient until the prosthetic heart valve is positioned within an annulus of the native heart valve.

In some examples, the method can include inflating a balloon, the inflating resulting in radial expansion of a prosthetic heart valve and engagement of the prosthetic heart valve with the annulus of the native heart valve.

In some examples, the method can delivery apparatus can include a restraining assembly that limits, restrains, and/or restricts axial elongation of the balloon during inflation thereof.

In some examples, the method includes detecting a fluid pressure reading to identify contact between the radially expanded prosthetic heart valve and the annulus of the native heart valve.

The above method(s) can be performed on a living animal or on a simulation, such as on a cadaver, cadaver heart, anthropomorphic ghost, simulator (for example, with body parts, heart, tissue, etc. being simulated).

In one representative example, a delivery assembly comprises: a radially expandable prosthetic valve comprising an annular frame and a valve structure disposed within the frame; a delivery apparatus comprising: a shaft; a balloon mounted on a distal end portion of the shaft, wherein the prosthetic valve is crimped in a radially compressed state around a central portion of the balloon, and wherein the balloon is inflatable to cause transition of the prosthetic valve from the radially compressed state to a radially expanded state; and a restraining assembly for limiting axial elongation of the balloon during inflation thereof, the restraining assembly comprising a proximal end cap extending over a proximal portion of the balloon and a distal end cap extending over a distal portion of the balloon, and a plurality of tethers extending from the proximal end cap to the distal end cap.

In another representative example, a medical apparatus for insertion within a patient's body comprises: a shaft; a balloon mounted around a distal end portion of the shaft; and a restraining assembly comprising a proximal end cap extending over a proximal portion of the balloon and a distal end cap extending over a distal portion of the balloon, and a plurality of tethers extending from the proximal end cap to the distal end cap.

In another representative example, a medical apparatus for insertion into a patient's body, the medical apparatus comprises: a shaft; an inflatable balloon mounted on a distal end portion of the shaft; and restraining means for limiting axial elongation of the balloon during inflation thereof.

In another representative example, a method of implanting a prosthetic heart valve utilizing a delivery apparatus comprises: introducing a distal end portion of the delivery apparatus into vasculature of a patient, the delivery apparatus comprising a handle portion, an elongated shaft extending from the handle portion, the distal end portion comprising an inflatable balloon mounted on the shaft, a prosthetic heart valve in a radially compressed state mounted over the balloon, and a restraining assembly, the restraining assembly comprising a proximal end cap extending over a proximal portion of the balloon and a distal end cap extending over a distal portion of the balloon, and a plurality of tethers extending from the proximal end cap to the distal end cap; advancing the distal end portion of the delivery apparatus toward a native heart valve of the patient until the prosthetic heart valve is positioned within an annulus of the native heart valve; and, after the prosthetic heart valve has been positioned in the native annulus, inflating the balloon, the inflating resulting in radial expansion of the prosthetic heart valve and engagement of the prosthetic heart valve with the annulus of the native heart valve.

In some examples, a delivery assembly, a medical apparatus, and/or a method can comprise one or more of the components and/or steps recited in Examples 1-48 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to one example.

FIG. 2 is a side view of an example of a delivery apparatus configured to deliver and implant a radially expandable prosthetic heart valve at an implantation site.

FIGS. 3-5 are enlarged, cross-sectional views of a distal end portion of a delivery apparatus showing inflation of a balloon for deployment of a prosthetic heart valve mounted on the balloon.

FIGS. 6-7 are schematic illustrations of an exemplary delivery apparatus including a balloon restraining assembly.

FIGS. 8-9 are schematic illustrations of another exemplary delivery apparatus including a balloon restraining assembly.

FIGS. 10-11 are a perspective view and an axial view, respectively, of another exemplary delivery apparatus include a balloon retraining assembly.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, “e.g.” means “for example,” and “i.e.” means “that is.”

As used herein, the term “substantially,” “about,” and “approximately” mean within a range of a given value, such as within a range of +/−20% of a given value, for example, within a range of +/−10% of a given value.

### Overview of the Disclosed Technology

Described herein are examples of a medical instrument or apparatus (in some examples referred to as a delivery catheter or delivery apparatus) that can be used to navigate a subject's vasculature to deliver an implantable, expandable medical device (for example, a prosthetic heart valve), tools, agents, or other therapy to a location within the body of a subject. Examples of procedures in which the catheters are useful include neurological, urological, gynecological, fertility (for example, in vitro fertilization, artificial insemination), laparoscopic, arthroscopic, transesophageal, transvaginal, transvesical, transrectal, and procedures including access in any body duct or cavity. Some examples include placing implants, including stents, grafts, embolic coils, and the like; positioning imaging devices and/or components thereof, including ultrasound transducers; positioning energy sources, for example, for performing lithotripsy, RF sources, ultrasound emitters, electromagnetic sources, laser sources, thermal sources, and the like; performing a valvuloplasty, angioplasty, or other remodeling procedures; and delivering agents, such as drugs, into a patient's body.

The following description proceeds with reference to delivery apparatuses with inflatable balloons for implanting a prosthetic heart valve. However, it should be understood that the disclosed delivery apparatuses can be used to implant various other medical devices, such as any of those described above. Moreover, the balloons described herein can be incorporated into medical apparatus that are used to perform procedures that do not involve implanting an implantable medical device, such as balloon catheters used to perform valvuloplasty or other similar procedures.

As introduced above, a delivery apparatus can include a balloon for radially expanding an implantable medical (for example, a prosthetic valve) at a target implantation site (for example, within an annulus of a native heart valve). For example, a distal end of the delivery apparatus including a radially compressed prosthetic valve mounted over a deflated balloon can be positioned within a native annulus. After the positioning, the balloon can be inflated to transition of the prosthetic valve from the radially compressed state to a radially expanded state.

In some examples, the balloon can be coupled to a shaft of a balloon catheter and can be in communication with a fluid source (for example, a syringe or other pressurized reservoir containing a fluid) and a pressure sensor (for example, a pressure gauge, a pressure transducer, a pressure transmitter, etc.) for sensing fluid pressure inside the balloon during inflation thereof. In some examples, the fluid pressure within in the balloon can be utilized to estimate a radial force of the prosthetic valve on the implantation site (for example, a force exerted on the native annulus). In some examples, the fluid pressure within in the balloon can be utilized to estimate and/or detect contact between the radially expanded prosthetic valve and the implantation site (for example, contact between the radially expanded prosthetic valve and the native annulus).

For example, at an initial phase of implantation where the prosthetic valve is expanding but has not yet come into contact with the native annulus, the fluid pressure within the balloon (detected by the pressure sensor) can remain relatively low and/or substantially constant as fluid flows into the balloon from the fluid source. In a subsequent phase of implantation where the radially expanded prosthetic valve comes into contact with the native annulus, further expansion of the valve and the balloon may be limited by the native anatomy. Therefore, flow of fluid into the balloon may cause the pressure in the balloon to be higher than in the initial phase and/or the pressure may increase at a faster rate relative to the initial phase.

In some examples, fluid pressure (for example, a pressure value, a change in pressure, and/or a rate of change of the pressure) can be utilized to estimate and/or identify contact between the prosthetic valve and the native annulus. For example, a pressure value, a change in pressure, and/or a rate of change in pressure can be compared to model values to estimate when contact between the radially expanded prosthetic valve and the native annulus occurs. In some examples, identification of the contact between the prosthetic valve and the native annulus can be utilized to determine when to initiate subsequent phases of implantation, such as, for example, stopping flow of fluid to the balloon, deflation of the balloon, withdraw of the balloon catheter, and/or deployment of other prosthetic valve features or implantable devices (such as, for example, deployment one or more anchors on a frame of the prosthetic valve, transitioning of leaflets from a delivery state to an active state, implantation of a secondary prosthetic device, etc.).

However, known or conventional delivery systems may have one or more issues that limit or hinder identification and/or estimation of contact between a prosthetic valve and native anatomy. For example, a delivery system can include a radially compressed prosthetic valve crimped over a center portion of a compliant balloon, and proximal and distal end portions of the balloon may be exposed from the crimped prosthetic valve. As fluid is delivered to the balloon, the proximal and distal end portions of the balloon may expand first, and expansion of the center portion may lag or otherwise be delayed relative to the end proximal and distal end portions. Further, the compliant balloon may axially elongate prior to radially expanding. Uneven (nonuniform) inflation at the proximal and distal end portions of the balloon relative to the center portion and/or axial elongation of the compliant balloon prior to radial expansion can introduce artifacts that make it harder to predict the force applied thereby on the surrounding native anatomy based on inflation pressure models (for example, models of pressure values, changes in pressure, and/or rates of change of pressure). Additionally, radially expansion of the prosthetic valve may occur unevenly. For example, inflow and outflow end portions of the prosthetic valve may radially expand prior to a center portion of the prosthetic valve radially expanding.

Further, native valve anatomy can vary between individuals. For example, native aortic annuluses can vary in size from about 18 mm to about 27 mm. A compliant balloon can have the advantage of being usable over a working range corresponding to, for example, the range in sizes of native annuluses, however, utilization of fluid pressure within the balloon to identify contact between a radially expanded prosthetic valve and the native annulus may be limited at the higher end of the working range (for example, as the prosthetic valve approached a maximum diameter and/or the balloon approaches maximum degree of inflation).

The delivery apparatus, assemblies, and methods disclosed herein can address one or more of the foregoing issues. In some examples, a delivery apparatus can include a balloon having one or more restraints (which can also be referred to as “restraining features” and “restraining assemblies”). For example, a delivery apparatus can include a balloon having one or more restraining assemblies that, during inflation of the balloon, restrain, restrict, and/or limit axial elongation and/or encourage radial expansion thereof. Further, in some examples, a delivery apparatus can include a balloon having one or more restraining assemblies that encourage uniform radial expansion of a prosthetic valve. In some examples, a delivery apparatus can include a balloon having one or more restraining assemblies that improve predictability of inflation and pressurization characteristics over a working range of the balloon (for example, improve predictability of inflation and pressurization characteristics at the higher end of the working range), and thereby improve accuracy in estimating and/or identifying contact between a radially expanded prosthetic valve and native anatomy at an implantation site.

In some examples, a delivery apparatus can include a compliant balloon having non-compliant (or less-compliant) proximal and distal sleeves or end caps that respectively cover proximal and distal ends of the balloon. One or more tethers can extend between and be coupled to the proximal and distal sleeves or end caps. In some examples, the tethers can have a length that restricts, retrains, and/or limits axial elongation of the balloon (that is, movement of the proximal and distal ends in a direction away from each other) past a desired and/or selected degree or distance. In some examples, a delivery apparatus can include a balloon having a compliant central portion and non-compliant (or less-compliant) proximal and distal end portions. In some examples, a compliant balloon can include a plurality of axially extending members (for example, axially extending metallic bars or strips) coupled to an outer surface of a center portion of the balloon, which can be angularly spaced around a circumference of the center portion. The axially extending members can improve uniformity in radial expansion of the balloon and thereby improve uniformity in radial expansion of the prosthetic valve overlying the balloon.

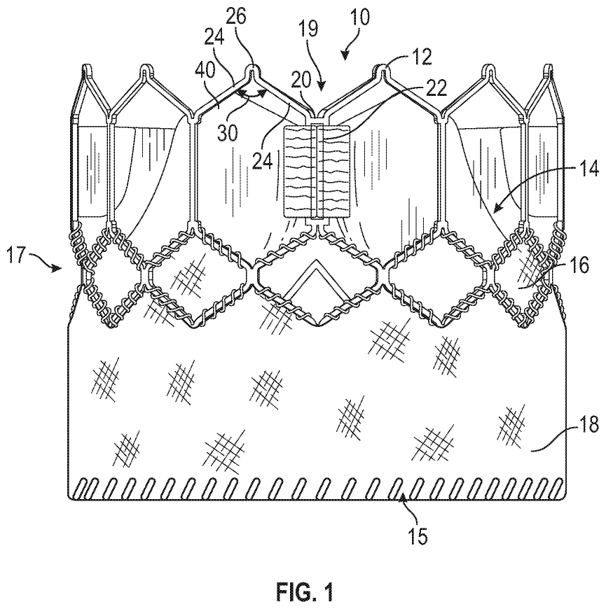

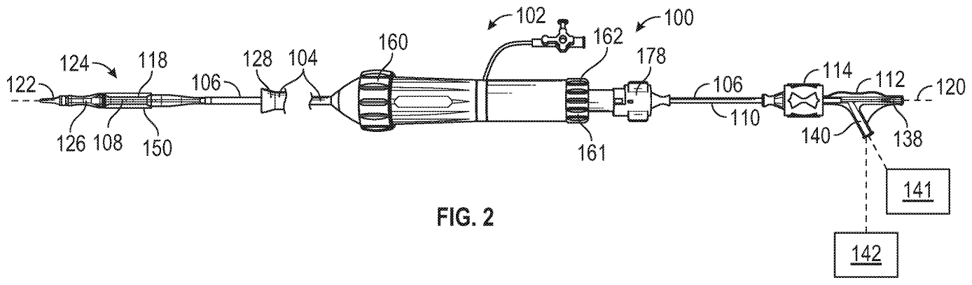

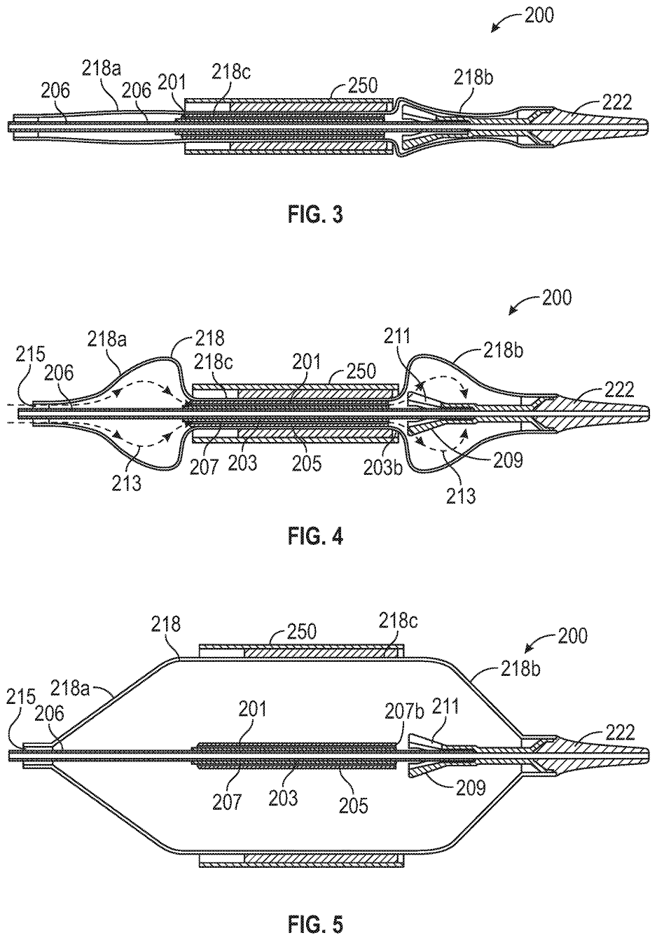

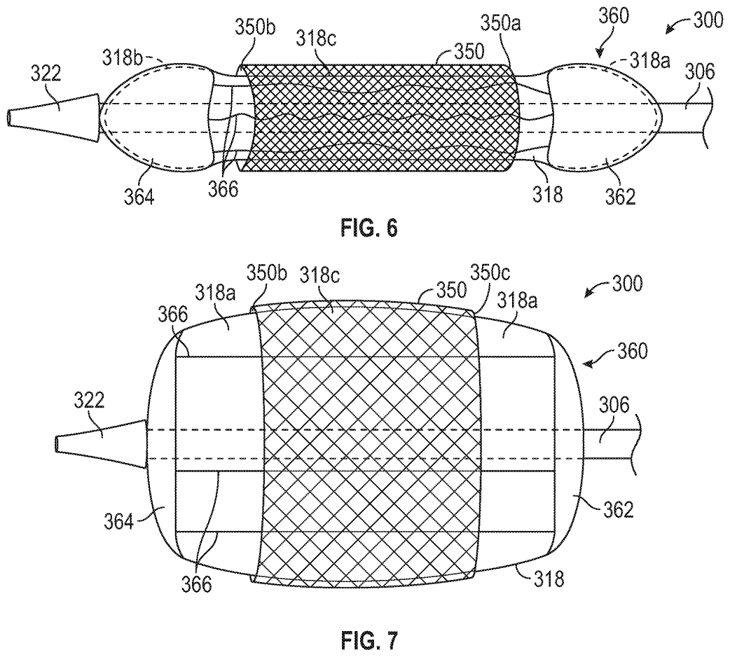

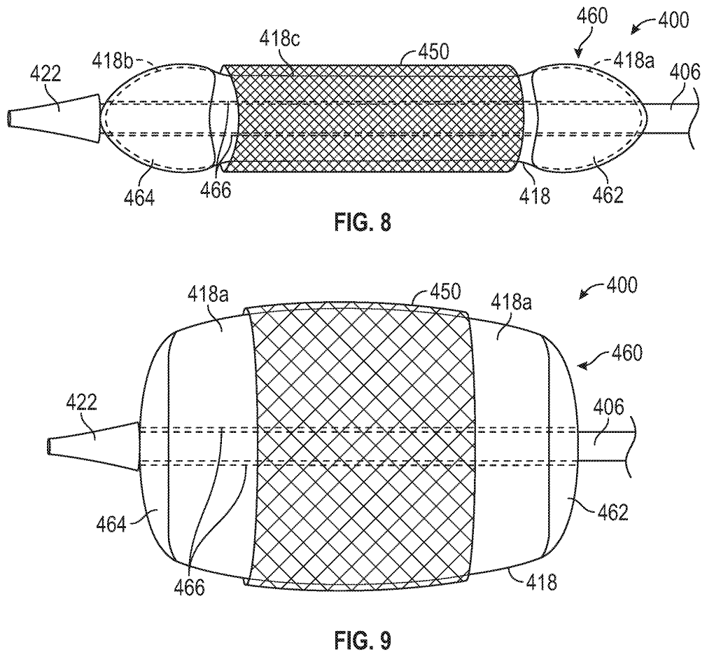

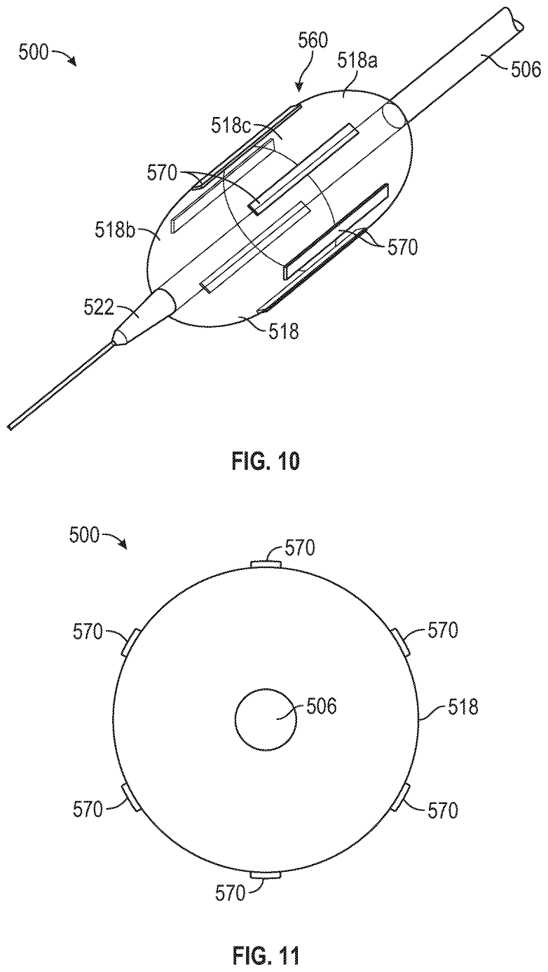

FIG. 1 shows an exemplary radially compressible and expandable prosthetic valve. FIG. 2 shows an exemplary delivery assembly for transcatheter delivery of a radially compressible and expandable prosthetic valve. FIGS. 3-5 illustrate exemplary phases of balloon inflation for radial expansion of a prosthetic valve from a radially compressed configuration to a radially expanded configuration. FIGS. 6 and 7 illustrate a first exemplary delivery apparatus including a balloon and a restraining assembly in accordance with the present disclosure. FIGS. 8 and 9 illustrate a second exemplary delivery apparatus including a balloon and a restraining assembly in accordance with the present disclosure. FIGS. 10 and 11 illustrate a third exemplary delivery apparatus including a balloon and a restraining assembly in accordance with the present disclosure. Although FIGS. 6-11 show first, second, and third exemplary delivery apparatus, it will be appreciated that the delivery apparatus disclosed herein can include one or more features of other delivery apparatus disclosed herein.

### Examples of the Disclosed Technology

FIG. 1 shows an exemplary prosthetic valve 10, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

As shown in FIG. 1, the prosthetic valve 10 can have four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19. The inner skirt 16 can be arranged on and/or coupled to an inner surface of the frame 12 while the outer skirt 18 can be arranged on and/or coupled to an outer surface of the frame 12.

The inner and/or outer skirts can be wholly or partly formed of any suitable biological material, synthetic material (for example, any of various polymers), or combinations thereof. In some examples, the skirt can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the skirt can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirt can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the skirt can comprise a sponge material or foam, such as polyurethane foam. In some examples, the skirt can comprise natural tissue, such as pericardium (for example, bovine pericardium, porcine pericardium, equine pericardium, or pericardium from other sources).

The valvular structure 14 can comprise three leaflets 40, collectively forming a leaflet structure, which can be arranged to collapse in a tricuspid arrangement, although in other examples there can be greater or fewer number of leaflets (for example, one or more leaflets 40). The leaflets 40 can be secured to one another at their adjacent sides to form commissures 22 of the valvular structure 14. The lower edge of valvular structure 14 can have an undulating, curved scalloped shape and can be secured to the inner skirt 16 by sutures (not shown). In some examples, the leaflets 40 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

The frame 12 can be radially compressible (collapsible) and expandable (for example, expanded configuration shown in FIG. 1) and comprise a plurality of interconnected struts 24. A plurality of apices 26 that are spaced circumferentially apart are formed at the inflow end portion 15 and the outflow end portion 19 of the frame 12 (only the apices 26 at the outflow end portion 19 are visible in FIG. 1). Each apex 26 is formed at a junction between two angled struts 24 at either the inflow end portion 15 or the outflow end portion 19. FIG. 1 depicts a known frame design with apices 26 that form a U-shaped bend between the two angled struts 24. In some examples, an angle 30 between the two angled struts 24, connected at the apex 26, can be in a range of 90 to 120 degrees.

The frame 12 can be formed with a plurality of circumferentially spaced slots, or commissure windows 20 that are adapted to mount the commissures 22 of the valvular structure 14 to the frame.

The frame 12 can be made of any of various suitable plastically-expandable materials (for example, stainless steel, etc.) or self-expanding materials (for example, Nitinol) as known in the art. When constructed of a plastically-expandable material, the frame 12 (and thus the valve 10) can be crimped to a radially compressed state on a delivery catheter and then expanded inside a patient by an inflatable balloon or equivalent expansion mechanism. When constructed of a self-expandable material, the frame 12 (and thus the valve 10) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 12) include, metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP 35N™/UNS R30035 comprises 35% nickel, 35% cobalt, 20% chromium, and 10% molybdenum, by weight.

FIG. 2 shows an example of a delivery apparatus 100 that can be used to implant an expandable prosthetic heart valve (for example, prosthetic heart valve 10 of FIG. 1 or others of the prosthetic heart valves described herein). In some examples, the delivery apparatus 100 is adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (for example, move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (for example, inflation fluid) from a fluid source 141 (for example, a syringe). In some examples, the second port 140 can be fluidly coupled to an inner lumen of the intermediate shaft 106 and can be configured for fluid communication with the fluid source 141. In some examples, the second port 140 is in communication with a pressure sensor 142 (for example, a pressure gauge, a pressure transducer, a pressure transmitter, etc.). The pressure sensor 142 can, for example, be mounted on a syringe that is used as the fluid source 141.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIG. 2), or to an alternate component at the distal end of the delivery apparatus 100 (for example, a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (for example, for fine positioning at the implantation site). Further details on the delivery apparatus 100 can be found in WIPO Publication No. WO2022/046585, which is incorporated by reference herein.

In some examples, an exemplary delivery apparatus for implantation of a prosthetic device (for example, a prosthetic heart valve) in the heart can include an elongated shaft comprising a proximal end portion and a distal end portion, an inflatable balloon, and a valve mounting member. The balloon can be mounted on the distal end portion of the shaft. The valve mounting member can be disposed on the distal end portion of the shaft within the balloon and can be configured to facilitate frictional engagement between the prosthetic heart valve and the balloon when the prosthetic heart valve is mounted in a radially compressed state on the balloon and surrounding the mounting member. The mounting member can comprise at least one longitudinally extending fluid passageway though which an inflation fluid in the balloon can flow.

Turning to FIGS. 3-5, a distal end portion of an exemplary delivery apparatus 200 including a mounting member 201 mounted on a balloon shaft 206 and having a fluid passageway for flow of an inflation fluid therethrough is shown in cross-sectional views. FIGS. 3-5 illustrate inflation of a balloon 218 for deployment of a prosthetic heart valve 250 mounted over the balloon 218 and the balloon shaft 206. It will be appreciated that the delivery apparatus 200 can include one or more features of the delivery apparatus 100 discussed above, and/or alternate or additional features, such as those of the delivery apparatuses including valve mounting members disclosed in U.S. Pat. No. 9,119,716, which is incorporated by reference herein.

As discussed above, the prosthetic valve 250 can be positioned at a target implant site or other deployment location while the prosthetic valve 250 is mounted in a radially compressed state on the balloon 218 and surrounding the mounting member 201 (FIG. 3). When the prosthetic valve 250 is at a deployment location, the balloon 218 can be inflated to expand the prosthetic valve 250 (that is, to transition the prosthetic valve 250 to a radially expanded state) so as to come into contact with a native annulus at the implantation site (FIGS. 4-5). In some examples, the expanded prosthetic valve 250 becomes anchored within the native aortic annulus by the radial outward force of the frame of the prosthetic valve against the surrounding tissue.

The mounting member 201 within the balloon can be configured to allow the inflation fluid (for example, saline) to flow unobstructed from the proximal end portion 218a of the balloon to the distal end 218b of the balloon. In some examples, the mounting member 201 can comprise a cylindrical inner wall 203 and a cylindrical outer wall 205 forming a channel 207 therebetween. In some examples, a plurality of angularly spaced ribs (for example, three ribs) can connect the inner and outer walls 203, 205 along a length of the mounting member 201 and the channel 207 can include a plurality of channels defined by the ribs. The inner wall 203 can be secured to the outer surface of the shaft 206 within the balloon 218. In some examples, the mounting member 201 can be made of a material (for example, polyurethane or another suitable plastic) that does not radially compress and/or resists radial compression when the prosthetic valve 250 is crimped onto the balloon 218. In some examples, a proximal portion 209 of the nose cone 222 also can be formed with one or more slots 211 (for example, three slots) that allow the inflation fluid to flow more easily past the proximal nose cone portion 209 into the distal section 218b of the balloon 218.

As shown in FIG. 4, during inflation of the balloon 218, inflation fluid can flow (in the direction of arrows 213) from a proximal opening 215 of the balloon 218 and into the proximal section 218a of the balloon, and can flow from the proximal section 218a through the channel 207 (between the inner and outer walls 203, 205 of the mounting member 201), through the one or more slots 211 in the proximal portion 209 of the nose cone 222, and into the distal section 218b of the balloon. Since the inflation fluid can pressurize and inflate the proximal and distal sections 218a, 218b of the balloon at substantially the same rate, the balloon 218 can be inflated for controlled expansion of the prosthetic valve. In some examples, expansion of a central portion or section 218c of the balloon 218 can lag or can be otherwise be delayed relative to expansion of the proximal and distal sections 218a, 218b. For example, as fluid flows into balloon 218 and the balloon is in a partially inflated state (FIG. 4), the proximal and distal end portions 218a, 218b of the balloon can have a greater diameter than the central portion 218c, whereas, in an inflated state of the balloon (FIG. 5), the central portion 218c can have a diameter that is greater than or equal to that of the proximal and distal end portions 218a, 218b. In another example, the balloon 218 may axially elongate prior to radially expanding.

As discussed above, uneven (nonuniform) inflation at the proximal and distal end portions of the balloon relative to the center portion and/or axial elongation of the compliant balloon can introduce artifacts that make it harder to predict the force applied thereby on the surrounding native anatomy based on inflation pressure models (for example, models of pressure values, changes in pressure, and/or rates of change of pressure). Additionally, radially expansion of the prosthetic valve may occur unevenly. For example, inflow and outflow end portions of the prosthetic valve may radially expand prior to a center portion of the prosthetic valve radially expanding.

In order to address the foregoing issues, the delivery apparatuses disclosed herein can include one or more restraints, restraining features, and/or restraining assemblies disposed on and/or over a balloon. Exemplary delivery apparatuses 300, 400, 500 including restraints and restraining assemblies are schematically illustrated in FIGS. 8-11. It will be appreciated that the delivery apparatuses 300, 400, 500 can include one or more of the features of the delivery apparatus 100, 200 or other additional or alternate features, such as those described in U.S. Pat. No. 9,119,716, previously incorporated herein. Also, the apparatuses 300, 400, 500 can be used for performing medical procedures other than delivering and implanting devices inside a patient's body.

Turning to FIGS. 6 and 7, a distal portion of the delivery apparatus 300 includes a balloon 318 mounted over a shaft 306 having an optional nose cone 322 at its distal end, and a prosthetic valve 350 mounted over a center portion 318c of the balloon 318. As in the examples described above, the balloon 318 can be a compliant or semi-compliant balloon configured to be inflated with a fluid to transition the prosthetic valve 350 from a radially compressed state (FIG. 6) to a radially expanded state (FIG. 7).

The compliance of a balloon refers to the ability of the balloon to expand from a predetermined increase in inflation pressure. Compliant and semi-compliant balloons typically respond to an increase in inflation pressure by initially increasing in diameter along sections of the balloon subject to less resistance (for example, the proximal and distal sections, which typically are not covered by the prosthetic valve), as depicted in FIGS. 3-5. Also, compliant and semi-compliant balloons typically can stretch radially and/or axially under inflation pressure, which allows a single balloon to be used to expand a prosthetic valve to a working diameter within a range of working diameters or to expand different size prosthetic valves to different working diameters. Non-compliant balloons typically expand uniformly along their longitudinal axis and exhibit little, if any, stretchability under normal inflation pressures, and therefore they typically cannot be inflated beyond a predetermined diameter.

The delivery apparatus 300 can further include a restraining assembly 360, which can include a proximal end cap 362 (also referred to a “proximal sleeve”), a distal end cap 364 (also referred to a “distal sleeve”), and a plurality of tethers 366 that extend from the proximal end cap 362 to the distal end cap 364. In some examples, the proximal end cap 362 covers and/or extends over a proximal portion 318a of the balloon, and the distal end cap 364 covers and/or extends over a distal portion 318b of the balloon.

In some examples, the proximal and distal end caps 362, 364 can be separate members that are attached to the balloon via, for example, suturing, an adhesive, and/or thermal bonding. In some examples, the end caps 362, 364 extend over the proximal and distal sections 318a, 318b but are not attached or bonded to the outer surface of the balloon. In some examples, the proximal and distal end caps 362, 364 can be comprised of the same material as the balloon 318 but can have a greater thickness than the material of the balloon 318, and therefore are less compliant than the balloon. In some examples, the proximal and distal end caps 362, 364 can comprise a non-compliant or less compliant material relative to the material of the balloon 318.

In some examples, the balloon 318 can be made of a first material, including, for example, any of various polyamides or co-polyamides, such as nylon (for example, nylon 12), Pebax®, Grilamid L25, or silicone and/or other materials or combinations thereof. In some examples, the end caps 362, 364 can be made of a second material that is less compliant than the first material. The second material can have a higher stiffness modulus than the first material and can include such materials as a polyethylene terephthalate (also known as PET), polybutylene terephthalate, or thermoplastic elastomer copolyester and/or other materials or combinations thereof.

In some examples, the end caps 362, 364 and the balloon can made of the same first material, or alternatively, the end caps 362, 364 and the balloons can be made of different materials that have the same or similar stiffness modulus. In such examples, the end caps can have a thickness (measured from an inner surface to an outer surface) that is greater than a thickness of the balloon. For example, the end caps can have a thickness in a range of, for example, 0.04 mm to 0.2 mm, and the material of the balloon can have a lesser thickness in a range of, for example, 0.03 mm to 0.15 mm.

In some examples, the end caps 262, 264 can comprise a braided, woven, or knitted structure, such as any of fabrics described above in connection with skirts 16, 18 of the prosthetic valve 10. In some examples, the braided, woven, or knitted structure of the end caps 262, 264 can be formed from metal wires. In some examples, the braided, woven, or knitted structure of the end caps 262, 264 exhibits little to no stretchability when subjected to the outward pressure of the balloon 318.

In some examples, the proximal and distal end caps 362, 364 can be integral (non-separate) portions of the balloon 318. In other words, the end caps 362, 364 can form the proximal and distal ends of the balloon 318. In some examples, the proximal and distal end caps 362, 364 can comprise the same material as the center portion 318c of the balloon but can have a greater thickness relative a wall of the center portion, thereby forming thicker proximal and distal end portions of the balloon 318. In other words, the balloon 318 can comprises a layer of material that has a greater thickness (measured from an inner surface to an outer surface) at the proximal and distal sections 318a, 318b compared to a thickness at the central section 318c. In another example, the proximal and distal end caps 362, 364 can comprise a non-compliant or less compliant material forming the proximal and distal ends of the balloon 318, and the center portion 318c can comprise a material that is more compliant relative to the material of the proximal and distal end caps 362, 364.

In some examples, the plurality of tethers 366 can be attached and/or coupled to (for example, adhered or sutured to, etc.) or can be integrally formed with and extend from a perimeter portion of each of the end caps 362, 364 and extend over an exterior surface of the balloon 318. In some examples, the plurality of tethers 366 can be angularly spaced around the perimeter portions of the end caps 362, 364. In some examples, the plurality of tethers 366 can include two or more tethers (such as, for example, three, four, five, six, seven, eight, etc. tethers). In some examples, a retraining assembly can include three to twenty tethers. In some examples, a restraining assembly can include six to ten tethers.

In some examples, the tethers can be comprised of one or more of the exemplary materials for the end caps discussed above (such as, for example, polyamides, co-polyamides, a braided, woven, or knitted structure, etc.). In some examples, the tethers 366 can be comprised of a high tensile strength biocompatible material, such as an ultra-high molecular weight polyethylene (UHMPE) force fiber®, polypropylene (for example, Prolene 4-0), polytetrafluoroethylene (PTFE), expanded PTFE (ePTFE), and/or other similar materials or combinations thereof. In some examples, the tethers can comprise wires or sutures (for example, braided sutures).

As can be seen in FIG. 6, in a delivery configuration of the delivery apparatus 300, the balloon 318 is in a compressed, deflated state and the prosthetic valve 350 is in a radially compressed (and axially elongated) state over the central portion 318c of the balloon 318. The end caps 362, 364 can also be in a compressed state, and can extend over and/or cover the compressed proximal and distal end portions 318a, 318b of the balloon 318. In some examples, the end caps 362, 364 can be folded; for example, the end caps can have longitudinally extending pleats or folds. In some examples, in the compressed state, the end caps 362, 364 can extend over a majority of the proximal and distal end portions 318a, 318b of the balloon 318. In some examples, in the compressed state, the end caps 362, 364 can extend over an entirety of the proximal and distal end portions 318a, 318b of the balloon 318. In some examples, in the compressed state, the proximal end cap 362 can be proximal of a proximal end 350a of the radially compressed prosthetic valve 350, and the distal end cap 364 can be distal of a distal end 350b of the radially compressed prosthetic valve 350 (as illustrated in FIG. 6). In some examples, portions (for example, the perimeter portions) of the compressed end caps 362, 364 can extend beneath the radially compressed valve 350.

When the delivery apparatus 300 is in the delivery configuration, the tethers 366 can be in a relaxed state. For example, the tethers 366 can have material slack (for example, the tethers are slackened along their length). In some examples, the relaxed tethers 366 can be free and/or loose over the exterior surface of the compressed balloon 318. In some examples, the relaxed tethers 366 can have a defined arrangement to, for example, prevent tangling or snagging of the tethers. For example, the relaxed tethers 366 can be folded, coiled, etc. over the exterior surface of the compressed balloon 318.

FIG. 7 shows the delivery apparatus 300 in a deployment configuration. As can be seen therein, the balloon 318 is in an inflated state and the prosthetic valve 350 is in a radially expanded (and axially foreshortened) state over the central portion 318c of the balloon 318. In some examples, the balloon 318 can be inflated similarly to the balloon 218 (illustrated in FIGS. 3-5 and described above), to cause transition of the prosthetic valve 350 from the radially compressed state to the radially expanded state. In some examples, inflation of the balloon 318 can differ from inflation of the balloon 218.

For example, as the proximal and distal end portions 318a, 318b (which are exposed from the radially compressed valve 350) are inflated, axial elongation of the balloon 318 can be restricted or limited by the restraining assembly 360. As the proximal and distal end portions 318a, 318b inflate, the end caps 362, 364 can expand, unfold, unfurl, deploy, and/or the perimeter portions thereof can move radially outward (for example, similar to deployment of a parachute), and the tethers 366 can be pulled taut between the perimeter portions of the end caps. As can be seen in FIG. 7, the tethers 366 can be moved radially outward relative to the shaft 306 by the balloon and a distance between each of the tethers 366 can be increased. In such examples, the tethers 366 can prevent or limit movement of the end caps 362, 364 away from each other, and thereby restrain, restrict, and/or limit axial elongation of the balloon 318 beyond a selected or desired distance. For example, a length of the tethers 366 can be selected to define the permitted axial elongation distance and/or the degree of permitted axial elongation of the inflated balloon 318.

As axial elongation of the balloon 318 is restricted and/or limited, the inflation fluid can be forced and/or directed into the center portion 318c of the balloon 318 and cause expansion of the center portion 318c. Thus, in some examples, the restraining assembly 360 can enable inflation of the center portion 318c of the balloon 318 at an earlier phase of the inflation process relative to delivery apparatus lacking the restraining assembly. In some examples, the restraining assembly 360 can enable more even (uniform) inflation of the proximal, distal, and center portions 318a, 318b, 318c of the balloon 318 relative to delivery apparatus lacking a restraining assembly. In some examples, the restraining assembly 360 can enable more symmetrical radial expansion of the prosthetic valve 350 when transitioning from the radially compressed state to the radially expanded state relative to a delivery apparatus lacking the restraining assembly. In some examples, the restraining assembly 360 can enable improved utilization of fluid pressure within the balloon 318 to identify contact between a radially expanded prosthetic valve 350 and a native annulus over the working range of the balloon and prosthetic valve (for example, improved utilization of fluid pressure a higher end of the working range) relative to a delivery assembly lacking the restraining assembly.

Turning to FIGS. 8 and 9, a distal end portion of a delivery assembly 400 is shown and described. The delivery apparatus 400 includes a balloon 418 mounted over a shaft 406 having an optional nose cone 422 at its distal end, and a prosthetic valve 450 mounted over a center portion 418c of the balloon 418. The delivery apparatus 400 can further include a restraining assembly 460, which can include a proximal end cap 462 covering and/or extending over a proximal portion 418a of the balloon, a distal end cap 464 covering and/or extending over a distal portion 418b of the balloon, and a plurality of tethers 466 that extend from the proximal end cap 462 to the distal end cap 464.

The delivery apparatus 400 can be similar to and have one or more features of the delivery apparatus 300 described above. For example, the restraining assembly 460 can have one or more features of the restraining assembly 360 described above. In some examples, the restraining assembly 460 can have one or more of the functions and/or advantages of the restraining assembly 360 described above. In some examples, the delivery apparatus 400 can differ from the delivery apparatus 300.

For example, the tethers 466 (rather than extending over an exterior surface of the balloon) can extend along the shaft 406 inside of the balloon 418. In such examples, the tethers 418 (rather than being attached to perimeter portions of the end caps) can be attached and/or coupled to or can be integrally formed with and extend from a central portion of each of the end caps 462, 464. As the tethers extend inside the balloon, in a delivery configuration of the delivery apparatus 400 (FIG. 8), the tethers 466 can be in a relaxed state, and can remain in a similar or same location when the delivery apparatus 400 is a deployed configuration (FIG. 9). In other words, as the end caps 462, 464 expand, unfold, unfurl, deploy, and/or the perimeter portions thereof move radially outward, the tethers 466 can stay in substantially a same location relative to the shaft 406 in the deployed configuration and can be pulled taut between the center portions of the end caps (for example, similar to deployment of an umbrella). In some examples, the tethers 466 can be taut (without any slack) when the balloon is in the deflated state.

In some examples, a delivery apparatus can include both tethers that extend over an exterior surface of a balloon (for example, the tethers 366) and tethers that extend interior of the balloon (for example, the tethers 466).

FIGS. 10 and 11 illustrate a distal end portion of another exemplary delivery apparatus 500, which includes a restraining assembly 560. In some examples, the restraining assembly 560 can be utilized in combination with another restraining assembly, such as, for example, either of the restraining assemblies 360, 460 discussed above. In some examples, the restraining assembly 560 can be utilized without an additional restraining assembly.

As can be seen in FIGS. 10 and 11, the distal portion of the delivery apparatus 500 includes a balloon 518 mounted over a shaft 506 having an optional nose cone 522 at its distal end. Although not shown, a prosthetic valve can be mounted over a center portion 518c of the balloon 518. As in the examples described above, the balloon 518 can be a compliant or semi-compliant balloon configured to be transitioned from a deflated state (not shown) to an inflated state (FIGS. 10 and 11) by filling the balloon with a fluid, which can cause radial expansion of a prosthetic valve crimped around the balloon.

The restraining assembly 560 includes a plurality of axial members 570 (which can also be referred to as “axial ribs”) attached to the balloon 518. In some examples, the plurality of axial members 570 can include two or more members (such as, for example, three, four, five, six, seven, eight, or more members). In the illustrated example, the restraining assembly 560 includes six axial members 570.

In some examples, the axial members 570 can be attached on an exterior surface of the balloon 518 (as in FIGS. 10 and 11). In some examples, the axial members 570 can be attached to an interior surface of the balloon 518. In some examples, the axial members 570 can be embedded in a wall of the balloon 518. In some examples, the axial members 570 extend over only a portion of the balloon. For example, the axial members 570 can extend over the central portion 518c of the balloon. In some examples, the axial members 570 can further extend over at least a section of each of the proximal and distal end portions 518a, 518b of the balloon. In some examples, the axial members 570 are angularly spaced around a circumference of the balloon 518.

In some examples, the axial members 570 can each comprise a strip or bar having a greater width than its height (as in FIGS. 10 and 11). In some examples, the axial members 570 can each comprise a wire or filament having a width that is equal to its height (for example, a wire having a circular cross-section or a wire with a square cross-section). In some examples, the axial members 570 can be formed from a non-compliant or less compliant material. For example, the axial members 570 can comprise a metal or metal alloy, such as cobalt chromium, steel, nitinol, and/or other metallic materials or combinations thereof. In some examples, the axial members 570 can comprise a polymer, such as high-density polyethylene (HDPE), ultra-high molecular weight polyethylene (UHMPE) force fiber®, polypropylene (for example, Prolene 4-0), polytetrafluoroethylene (PTFE), expanded PTFE (ePTFE), or other similar materials or combinations thereof. In some examples, the axial members 570 can comprise a combination of one or more of the foregoing materials. For example, the axial members 570 can comprise metallic strips with a polymer coating.

The axial members 570 can add structural support, strength, and/or rigidity to the central portion 518c of the balloon. Accordingly, in some examples, during inflation of the balloon 518, the center portion 518c can expand more evenly over its length. In some examples, the center portion 518c can expand more evenly relative to the proximal and distal portions 518a, 518b of the balloon 518. In some examples, lag or delay of expansion of the central portion 518c relative to the proximal and distal portions 518a, 518b can be reduced.

In some examples, the restraining assembly 560 can have one or more of the functions and/or advantages of the restraining assembly 360 described above. For example, the restraining assembly 560 can enable more even radial expansion of a prosthetic valve (for example, the center portion of the prosthetic valve can radially expand contemporaneously with the proximal and distal ends of the prosthetic valve) when transitioning from the radially compressed state to the radially expanded state relative to a delivery apparatus lacking the restraining assembly.

### Exemplary Methods

An exemplary method of implanting a radially compressible and expandable prosthetic device (for example, a prosthetic heart valve) in the heart using the delivery apparatus disclosed herein (for example, the delivery apparatus 300, 400, 500) comprises: (a) introducing a delivery apparatus into the body of a patient, the delivery device comprising a handle portion, an elongated shaft extending from the handle portion, the shaft having a distal end portion having an inflatable balloon and a prosthetic heart valve in a radially compressed state mounted thereon, the distal end portion additionally including a restraining assembly comprising proximal and distal end caps respectively extending over proximal and distal end portions of the balloon and a plurality of tethers extending between and attached to the end caps; (b) advancing the distal end portion of the delivery apparatus toward the native heart valve until the prosthetic valve is within or adjacent the annulus of the native heart valve; (c) positioning the prosthetic heart valve at a desired implantation position within the annulus of the native heart valve (for example, by rotating an adjustment device coupled to the handle portion and the shaft to cause the shaft and the prosthetic valve to move distally and/or proximally relative to the handle portion until the prosthetic heart valve is at the desired implantation position); and (d) after the prosthetic heart valve has been moved to the desired implantation position, inflating the balloon having the restraining assembly mounted thereon (which can, for example, restrain, restrict, and/or limit axial elongation of the balloon during inflation and/or improve even (uniform) radial expansion of the center portion of the balloon relative the proximal and distal end portions of the balloon during inflation), the inflating resulting in and/or causing the prosthetic heart valve to radially expand and engage the annulus of the native heart valve. In some examples, the method can further include detecting a fluid pressure of the balloon to identify contact between the radially expanded prosthetic valve and the native annulus.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery assembly comprising: a radially expandable prosthetic valve comprising an annular frame and a valve structure disposed within the frame; a delivery apparatus comprising: a shaft; a balloon mounted on a distal end portion of the shaft, wherein the prosthetic valve is crimped in a radially compressed state around a central portion of the balloon, and wherein the balloon is inflatable to cause transition of the prosthetic valve from the radially compressed state to a radially expanded state; and a restraining assembly for limiting axial elongation of the balloon during inflation thereof, the restraining assembly comprising a proximal end cap extending over a proximal portion of the balloon and a distal end cap extending over a distal portion of the balloon, and a plurality of tethers extending from the proximal end cap to the distal end cap.

Example 2. The delivery assembly of any example disclosed herein, particularly example 1, wherein the end caps are less compliant than the balloon.

Example 3. The delivery assembly of any example disclosed herein, particularly examples 1 or 2, wherein the proximal and distal end caps comprise a first material and the balloon comprises a second material, and wherein the second material is more compliant than the first material.

Example 4. The delivery assembly of any example disclosed herein, particularly example 3, wherein the first material comprises one or more of nylon, Pebax®, Grilamid L25, or silicone.

Example 5. The delivery assembly of any example disclosed herein, particularly examples 3 or 4, wherein the second material comprises one or more of polyethylene terephthalate (PET), polybutylene terephthalate, or thermoplastic elastomer copolyester.

Example 6. The delivery assembly of any example disclosed herein, particularly example 1, wherein each of the proximal and distal end caps comprises a braided, woven, or knitted structure.

Example 7. The delivery assembly of any example disclosed herein, particularly example 6, wherein the braided, woven, or knitted structure comprises one or more of yarns or metal wires.

Example 8. The delivery assembly of any example disclosed herein, particularly examples 1-7, wherein each of the proximal and distal end caps has a thickness that is greater than a thickness of a wall of the balloon.

Example 9. The delivery assembly of any example disclosed herein, particularly example 8, wherein the thickness of the wall of the balloon is in a range of 0.03 mm to 0.15 mm.

Example 10. The delivery assembly of any example disclosed herein, particularly examples 6 or 7, wherein the thickness of each of the proximal and distal end caps is in a range of 0.04 mm to 0.2 mm.

Example 11. The delivery assembly of any example disclosed herein, particularly examples 1-10, wherein the tethers extend over an exterior surface of the balloon.

Example 12. The delivery assembly of any example disclosed herein, particularly example 11, wherein a first end portion of each of the tethers is attached to a perimeter portion of the proximal end cap, and wherein a second end portion of each of the tethers is attached to a perimeter portion of the distal end cap.

Example 13. The delivery assembly of any example disclosed herein, particularly examples 11 or 12, wherein the tethers are angularly spaced around a circumference of the balloon.

Example 14. The delivery assembly of any example disclosed herein, particularly examples 1-10, wherein the tethers extend through an interior of the balloon.

Example 15. The delivery assembly of any example disclosed herein, particularly example 14, wherein a first end portion of each of the tethers is attached to a central region of the proximal end cap, and wherein a second end portion of each of the tethers is attached to a central region of the distal end cap.

Example 16. The delivery assembly of any example disclosed herein, particularly examples 14 or 15, wherein the tethers are angularly spaced around a circumference of the shaft.

Example 17. The delivery assembly of any example disclosed herein, particularly examples 1-16, wherein the tethers comprise one or more of nylon, Pebax®, Grilamid L25, silicone, polyethylene terephthalate (PET), polybutylene terephthalate, or thermoplastic elastomer copolyester, an ultra-high molecular weight polyethylene (UHMPE) force fiber®, polypropylene, polytetrafluoroethylene (PTFE), expanded PTFE (ePTFE), polyetheretherketone (PEEK), or polyurethane.

Example 18. The delivery assembly of any example disclosed herein, particularly examples 1-17, wherein the plurality of tethers comprises three to twenty tethers.

Example 19. The delivery assembly of any example disclosed herein, particularly examples 1-18, wherein the plurality of tethers comprises six to ten tethers.

Example 20. The delivery assembly of any example disclosed herein, particularly examples 1-19, further comprising an additional restraining assembly comprising a plurality of axial members attached to and extending over the central portion of the balloon.

Example 21. The delivery assembly of any example disclosed herein, particularly example 20, wherein the axial members comprise a metallic material.

Example 22. The delivery assembly of any example disclosed herein, particularly examples 20 or 21, wherein the axial members comprise a polymer.

Example 23. The delivery assembly of any example disclosed herein, particularly examples 20-22, wherein the axial members are angularly spaced around a circumference of the central portion of the balloon.

Example 24. The delivery assembly of any example disclosed herein, particularly examples 1-23, wherein the balloon is configured to be in communication with a fluid source and a pressure sensor for indicating a fluid pressure within the balloon.

Example 25. A medical apparatus for insertion within a patient's body, the medical apparatus comprising: a shaft; a balloon mounted around a distal end portion of the shaft; and a restraining assembly comprising a proximal end cap extending over a proximal portion of the balloon and a distal end cap extending over a distal portion of the balloon, and a plurality of tethers extending from the proximal end cap to the distal end cap.

Example 26. The medical apparatus of any example disclosed herein, particularly example 25, wherein the balloon comprises a first material and the end caps comprises a second material, and wherein the first material is more compliant than the second material.

Example 27. The medical apparatus of any example disclosed herein, particularly examples 25 or 26, wherein the second material has a greater stiffness modulus than the first material.

Example 28. The medical apparatus of any example disclosed herein, particularly examples 26-27, wherein the first material comprises one or more of one or more of nylon, Pebax®, Grilamid L25, or silicone.

Example 29. The medical apparatus of any example disclosed herein, particularly examples 26-28, wherein the second material comprises one or more of polyethylene terephthalate (PET), polybutylene terephthalate, or thermoplastic elastomer copolyester.

Example 30. The medical apparatus of any example disclosed herein, particularly example 25, wherein each of the proximal and distal end caps comprises a braided, woven, or knitted structure.

Example 31. The medical apparatus of any example disclosed herein, particularly example 30, wherein the braided, woven, or knitted structure comprises one or more of yarns or metal wires.

Example 32. The medical apparatus of any example disclosed herein, particularly examples 25-31, wherein each of the proximal and distal end caps has a greater thickness than a thickness of a wall of the balloon.

Example 33. The medical apparatus of any example disclosed herein, particularly example 32, wherein the thickness of the wall of the balloon is in a range of 0.03 mm to 0.15 mm.

Example 34. The medical apparatus of any example disclosed herein, particularly examples 32 or 33, wherein the thickness of each of the proximal and distal end caps is in a range of 0.04 mm to 0.2 mm.

Example 35. The medical apparatus of any example disclosed herein, particularly examples 25-34, wherein each tether has a first end portion attached to the proximal end cap and a second end portion attached to the distal end cap.

Example 36. The medical apparatus of any example disclosed herein, particularly example 35, wherein the first end portion of each of the tethers is attached to a perimeter portion of the proximal end cap, and wherein the second end portion of each of the tethers is attached to a perimeter portion of the distal end cap.

Example 37. The medical apparatus of any example disclosed herein, particularly examples 35 or 36, wherein the tethers extend over an exterior surface of the balloon.

Example 38. The medical apparatus of any example disclosed herein, particularly example 35, wherein the first end portion of each of the tethers is attached to a central region of the proximal end cap, and wherein the second end portion of each of the tethers is attached to a central region of the distal end cap.

Example 39. The medical apparatus of any example disclosed herein, particularly examples 35 or 38, wherein the tethers extend through an interior of the balloon.

Example 40. The medical apparatus of any example disclosed herein, particularly examples 25-39, wherein the tethers comprise one or more of nylon, Pebax®, Grilamid L25, silicone, polyethylene terephthalate (PET), polybutylene terephthalate, or thermoplastic elastomer copolyester, an ultra-high molecular weight polyethylene (UHMPE) force fiber®, polypropylene, polytetrafluoroethylene (PTFE), expanded PTFE (ePTFE), polyetheretherketone (PEEK), or polyurethane.

Example 41. The medical apparatus of any example disclosed herein, particularly examples 25-40, wherein the plurality of tethers comprises three to twenty tethers.

Example 42. The medical apparatus of any example disclosed herein, particularly examples 25-41, wherein the plurality of tethers comprises six to ten tethers.

Example 43. The medical apparatus of any example disclosed herein, particularly examples 25-42, further comprising an additional restraining assembly comprising a plurality of axial members attached to and angularly spaced around a circumference of the central portion of the balloon.

Example 44. A method of implanting a prosthetic heart valve utilizing a delivery apparatus, the method comprising: introducing a distal end portion of the delivery apparatus into vasculature of a patient, the delivery apparatus comprising a handle portion, an elongated shaft extending from the handle portion, the distal end portion comprising an inflatable balloon mounted on the shaft, a prosthetic heart valve in a radially compressed state mounted over the balloon, and a restraining assembly, the restraining assembly comprising a proximal end cap extending over a proximal portion of the balloon and a distal end cap extending over a distal portion of the balloon, and a plurality of tethers extending from the proximal end cap to the distal end cap; advancing the distal end portion of the delivery apparatus toward a native heart valve of the patient until the prosthetic heart valve is positioned within an annulus of the native heart valve; and, after the prosthetic heart valve has been positioned in the native annulus, inflating the balloon, the inflating resulting in radial expansion of the prosthetic heart valve and engagement of the prosthetic heart valve with the annulus of the native heart valve.

Example 45. The method of any example disclosed herein, particularly example 44, wherein the restraining assembly restricts axial elongation of the balloon during the inflating of the balloon.

Example 46. The method of any example disclosed herein, particularly examples 44 or 45, further comprising detecting a fluid pressure reading to identify contact between the radially expanded prosthetic heart valve and the annulus of the native heart valve.

Example 47. A medical apparatus for insertion into a patient's body, the medical apparatus comprising: a shaft; an inflatable balloon mounted on a distal end portion of the shaft; and restraining means for limiting axial elongation of the balloon during inflation thereof.

Example 48. The medical apparatus of any example disclosed herein, particularly example 47, wherein the restraining means comprises a proximal sleeve disposed in a proximal end portion of the balloon, a distal sleeve disposed in a distal end portion of the balloon, and a plurality of tethers extending from the proximal sleeve to the distal sleeve.

The features described herein with regard to any example can be combined with other features described in any one or more of the other examples, unless otherwise stated. For example, any one or more of the features of delivery apparatus can be combined with any one or more features of another delivery apparatus. As another example, any one or more features of restraining assembly can be combined with any one or more features of another restraining assembly.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A delivery assembly comprising:
a radially expandable prosthetic valve comprising an annular frame and a valve structure disposed within the frame;
a delivery apparatus comprising:
a shaft;
a balloon mounted on a distal end portion of the shaft, wherein the prosthetic valve is crimped in a radially compressed state around a central portion of the balloon, and wherein the balloon is inflatable to cause transition of the prosthetic valve from the radially compressed state to a radially expanded state; and
a restraining assembly for limiting axial elongation of the balloon during inflation thereof, the restraining assembly comprising a proximal end cap extending over a proximal portion of the balloon and a distal end cap extending over a distal portion of the balloon, and a plurality of tethers extending from the proximal end cap to the distal end cap.

2. The delivery assembly of claim 1, wherein the proximal and distal end caps are less compliant than the balloon.

3. The delivery assembly of claim 1, wherein the proximal and distal end caps comprise a first material and the balloon comprises a second material, and wherein the second material is more compliant than the first material.

4. The delivery assembly of claim 3, wherein the first material comprises one or more of nylon, Pebax®, Grilamid L25, or silicone.

5. The delivery assembly of claim 3, wherein the second material comprises one or more of polyethylene terephthalate (PET), polybutylene terephthalate, or thermoplastic elastomer copolyester.

6. The delivery assembly of claim 1, wherein each of the proximal and distal end caps comprises a braided, woven, or knitted structure.

7. The delivery assembly of claim 1, wherein each of the proximal and distal end caps has a thickness that is greater than a thickness of a wall of the balloon.

8. The delivery assembly of claim 1, wherein the tethers extend over an exterior surface of the balloon.

9. The delivery assembly of claim 8, wherein a first end portion of each of the tethers is attached to a perimeter portion of the proximal end cap, and wherein a second end portion of each of the tethers is attached to a perimeter portion of the distal end cap.

10. The delivery assembly of claim 1, wherein the tethers extend through an interior of the balloon.

11. The delivery assembly of claim 10, wherein a first end portion of each of the tethers is attached to a central region of the proximal end cap, and wherein a second end portion of each of the tethers is attached to a central region of the distal end cap.

12. The delivery assembly of claim 1, further comprising an additional restraining assembly comprising a plurality of axial members attached to and extending over the central portion of the balloon.

13. A medical apparatus for insertion within a subject's body, the medical apparatus comprising:
a shaft;
a balloon mounted around a distal end portion of the shaft; and
a restraining assembly comprising a proximal end cap extending over a proximal portion of the balloon and a distal end cap extending over a distal portion of the balloon, and a plurality of tethers extending from the proximal end cap to the distal end cap.

14. The medical apparatus of claim 13, wherein the balloon comprises a first material and the proximal and distal end caps comprises a second material, and wherein the first material is more compliant than the second material.

15. The medical apparatus of claim 13, wherein the second material has a greater stiffness modulus than the first material.

16. The medical apparatus of claim 13, wherein each of the proximal and distal end caps comprises a braided, woven, or knitted structure.

17. The medical apparatus of claim 13, wherein each of the proximal and distal end caps has a greater thickness than a thickness of a wall of the balloon.

18. A method of implanting a prosthetic heart valve utilizing a delivery apparatus, the method comprising:
introducing a distal end portion of the delivery apparatus into vasculature of a subject, the delivery apparatus comprising a handle portion, an elongated shaft extending from the handle portion, the distal end portion comprising an inflatable balloon mounted on the shaft, a prosthetic heart valve in a radially compressed state mounted over the balloon, and a restraining assembly, the restraining assembly comprising a proximal end cap extending over a proximal portion of the balloon and a distal end cap extending over a distal portion of the balloon, and a plurality of tethers extending from the proximal end cap to the distal end cap;
advancing the distal end portion of the delivery apparatus toward a native heart valve of the subject until the prosthetic heart valve is positioned within an annulus of the native heart valve; and
after the prosthetic heart valve has been positioned in the native annulus, inflating the balloon, the inflating resulting in radial expansion of the prosthetic heart valve and engagement of the prosthetic heart valve with the annulus of the native heart valve.

19. The method of claim 18, wherein the restraining assembly restricts axial elongation of the balloon during the inflating of the balloon.

20. The method of claim 18, further comprising detecting a fluid pressure reading to identify contact between the radially expanded prosthetic heart valve and the annulus of the native heart valve.
